(19) United States
(12) Patent Application Publication
(10) Pub. No.: US 2014/0081237 A1
(43) Pub. Date: Mar. 20, 2014

(54) HAND-HELD MICRO-LIPOSUCTION ADIPOSE HARVESTER, PROCESSOR, AND CELL CONCENTRATOR

(71) Applicant: TISSUE GENESIS, INC., Honolulu, HI (US)

(72) Inventors: Rolf Wolters, Kailua, HI (US); Anthony Yang, Honolulu, HI (US); Josh Nelson, Kaleohe, HI (US); Stuart K. Williams, Harrods Creek, KY (US)

(73) Assignee: TISSUE GENESIS, INC., Honolulu, HI (US)

(21) Appl. No.: 13/844,548

(22) Filed: Mar. 15, 2013

Related U.S. Application Data
(60) Provisional application No. 61/703,742, filed on Sep. 20, 2012.

(51) Int. Cl. A61M 1/00

(52) U.S. Cl. CPC A61M 1/0056
USPC 604/506; 604/140; 604/113

(57) ABSTRACT

Devices and methods are provided for aspirating adipose tissue with a portable device. This device may include a processing chamber, a cannula, a vacuum source, a filter or screen for separating connective tissue strands from adipose tissue, a digestion area, and a product cell concentration chamber.

### PRIORITY CLAIM

This application claims the benefit of provisional patent application Ser. No. 61/703,742, filed Sep. 20, 2012, titled “Hand-Held Micro-Liposuction Adipose Harvester, Processor, and Cell Concentrator”, the contents which are incorporated herein by reference in their entirety.

### TECHNICAL FIELD

This disclosure relates to the collection of adipose lipoaspirate, the subsequent processing of said aspirate, and the concentration of product resulting from said processing in an integrated system.

### BACKGROUND

Adipose tissue has numerous uses. When such tissue is digested, the freed cells are capable of a multitude of uses, including tissue engineering, tissue repair, release of therapeutic factors by the cell, including factors released as a result of genetic engineering of the cells.

A problem with existing methods of extracting and isolating adipose cells is that existing equipment, such as liposuction cannulas and pumps and digestion devices, is not expected to be very portable. Furthermore, the liposuction, digestion, and cell concentration are often carried out in separate devices, and there are consequent issues in maintaining sterility as tissue and cells are transferred between devices. Consequently, it would be advantageous to have a portable, sterile device which was capable of carrying out the functions of extracting tissue, digesting the tissue, and concentrating the cells.

### BRIEF SUMMARY

Described herein are various inventions, particular examples of which are summarized here. In one embodiment, device for harvesting, processing, and delivering a cell product is provided, comprising a portable unit comprising: a processing chamber; a cannula with one end connected to the processing chamber in an airtight manner, and the other end open to the atmosphere; a vacuum source connected to the processing chamber for creating a vacuum in the processing chamber when the vacuum source is activated, such that when the cannula is inserted into adipose tissue and the vacuum source is activated, the adipose tissue can be aspirated through the cannula into the processing chamber; a connective tissue filter or screen within the processing chamber with a first side and a second side, suitable for separating connective tissue strands from adipose tissue which may arrive into the processing chamber through the cannula, wherein the connective tissue remains in the vicinity of the first side of the connective tissue filter or screen, and a composition comprising adipose cells is allowed to pass to the second side of the connective tissue filter or screen.

In another embodiment, a method is described for aspirating adipose tissue, comprising: providing a portable device as described above, including any of its variations; inserting a tip of the cannula into adipose tissue within a patient; activating the vacuum source to induce a vacuum within the processing chamber, thereby causing a portion of the adipose tissue to move through the cannula into the processing chamber; and in the processing chamber, separating connective tissue strands from said portion of the adipose tissue, such that the remaining adipose tissue passes to the second side of the connective tissue filter or screen.

In a further embodiment, a method is described for aspirating adipose tissue, comprising: providing a portable device comprising a cannula connected to a chamber, wherein the chamber comprises a pressure outlet in fluid communication with a valve, an wherein the valve is in fluid communication with an evacuated vessel; inserting a tip of the cannula into adipose tissue within a patient; and opening the valve to induce a vacuum within the chamber, thereby causing a portion of the adipose tissue to move through the cannula into the chamber.

Various additional embodiments, including additions and modifications to the above embodiments, are described herein.

### BRIEF DESCRIPTION OF THE DRAWINGS

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification or the common knowledge within this field.

In the drawings:

FIG. 1 is an illustration of an example hand-held micro-liposuction fat harvester, processor, and purifier.

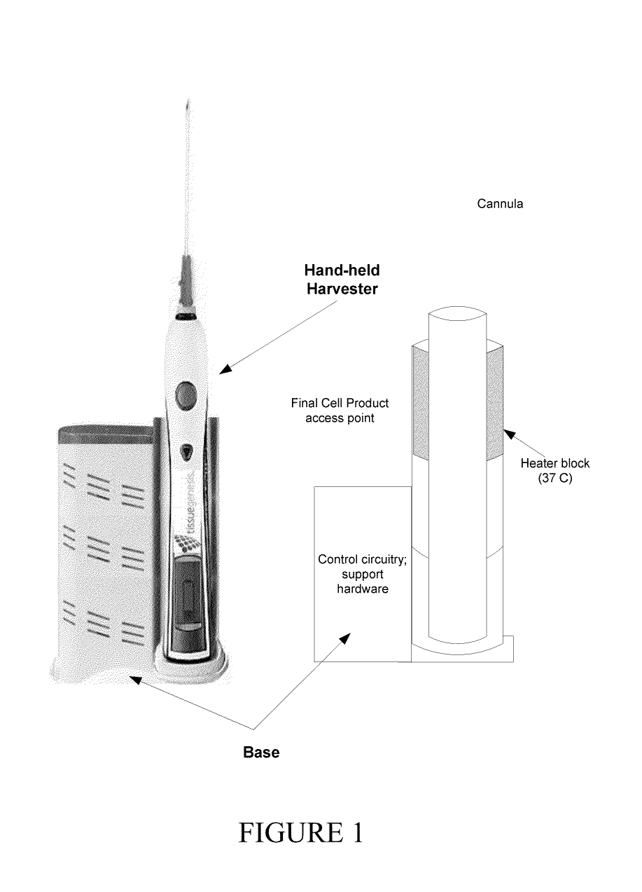

FIG. 2 is an illustration of a hand-held unit.

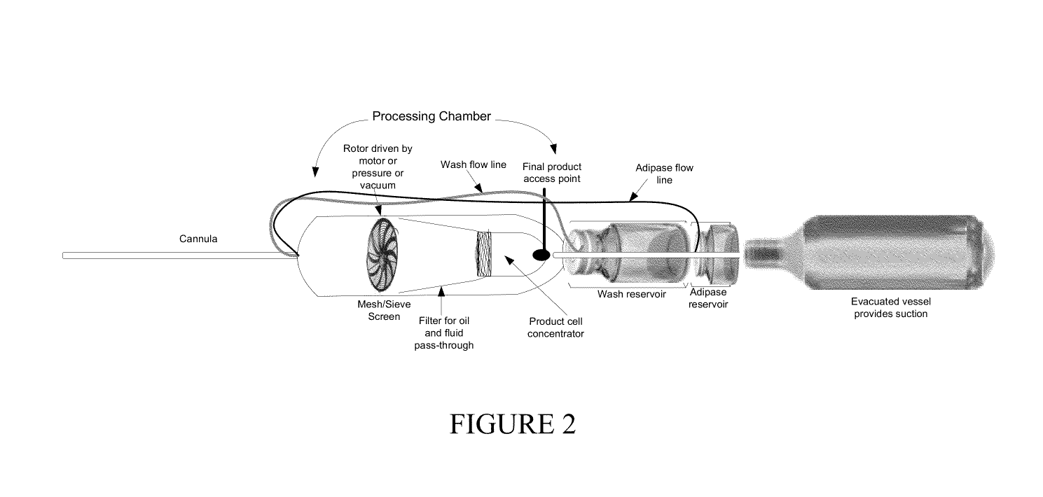

### DETAILED DESCRIPTION

Various example embodiments of the present inventions are described herein in the context of forming cell aggregates.

Those of ordinary skill in the art will realize that the following detailed description is illustrative only and is not intended to be in any way limiting. Other embodiments will readily suggest themselves to such skilled persons having the benefit of this disclosure.

In the interest of clarity, not all of the routine features of the implementations described herein are shown and described. In the development of any such actual implementation, numerous implementation-specific decisions must be made in order to achieve the developer's specific goals, such as compliance with application, safety, regulatory, and business constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Described herein are examples of an all-in-one hand-held device that harvests, processes and delivers a cell product. Novel features may include the following: a cordless hand-held liposuction device; the use of a hollow-fiber filter to separate SVF; the use of an evacuated chamber to supply vacuum for liposuction; the use of pressurized gas to move fluids for stem cell processing; a carousel of hand-held units that provides the opportunity to collect/process incremental volumes of fat depending on need (i.e. 3 units for example may provide 30 ml of processing); and enabling user selectable cell product concentration.

Advantages include the result that minimally invasive liposuction is user friendly and rapid, and can be done by any doctor, not just plastic surgeons. This has significant appeal. Fat-harvesting can also occur from multiple areas on the body of the patient (using multiple devices). Also, the cost of the device/disposable is very low compared to competitive approaches. Furthermore, dialing in the amount of fat needed and used depending on stem cell application is appealing (take only what you need)

In one embodiment of the inventions described herein, processing may involve first mechanically trapping and isolating connective tissue, allowing the remaining tissue to undergo further processing via enzymatic digestion, ultimately yielding the Stromal Vascular Fraction (SVF) including adipose derived stem cells (ADSC). The SVF may then be washed and concentrated by removing fluid and waste.

A preferred embodiment of the invention is that the integrated system is miniaturized and hand-hand, and that the volume of adipose tissue harvested is less than approximately 20 ml, however larger sizes would also be possible.

Embodiments described herein may be designed to aseptically aspirate and process adipose tissue in one continuous cycle. First, the device may utilize a vacuum to create adequate negative pressure through a cannula such that adipose tissue with which the cannula comes in contact is forcibly removed from its host and is collected by the device. Once within the device, the tissue encounters a mechanism that catches and holds connective tissue strands that may accompany the harvested adipose tissue. This mechanism can allow for the essentially connective tissue free remainder to pass into a chamber in which a series of wash and enzymatic digestion cycles may process the tissue under appropriate conditions. Upon completion, the suspension may be purified and concentrated, leaving behind a highly dense mass of cells. These cells may then be diluted according to values pre-determined by the operator.

In a preferred embodiment, vacuum can be provided by an accompanying pump mechanism connected by a hose. An alternate embodiment involves the incorporation of an evacuated vessel of sufficient volume to provide necessary suction. In another embodiment, a vacuum pump in the base evacuates a vessel in the hand-held while it is cradled in the base (pumps it down) so that the hand-held can operate independent of the base, providing vacuum for the lipoaspiration procedure. In one embodiment, vacuum is engaged by a push of a button on the handle by the operator. Vacuum is disengaged upon the release of said button. One embodiment contains a pressure vessel containing a supply gas, for example compressed air, oxygen or nitrogen. This gas supplies the positive pressure to move fluids throughout the system. An alternate embodiment produces positive pressure through the use of an incorporated micro-pump driven by a battery. In another embodiment, a pump in the base pressurizes a vessel in the hand-held while it is cradled in the base, so that the hand-held can operate independent of the base. In another embodiment, a pump in the base supplies positive pressure for the hand-held only while it is cradled, thereby only moving fluids by pressure during the cradled phase. Pressurized fluid flow is managed by the cycling of valves, including check valves, electro-magnetically actuated valves, and other valves.

Wash fluid and lyophilized enzymes may be supplied in specified aseptic vials (supply vials), and may be introduced into the device by “snapping-in” the vials in specified locations, during which incorporated vial access spikes puncture their respective seals. These supply vials may remain within the device for the duration of the process. The lyophilized enzyme may automatically be re-suspended by the device, as needed.

The device may be comprised of durable components and of disposable components. Any component that comes in contact with fluid is disposable. In one embodiment, the handle is comprised of an exoskeleton into which the disposables mount, micro-circuitry, and a small battery. In addition to the supply vials, the processing chamber in the handle and the vacuum trap/waste container that is mounted in the accompanying pump house are disposables and are connected via a disposable hose. An alternate embodiment has the entire hand-held as a disposable, into which the processing chamber, supply vials, and the pressure and vacuum vessels may be mounted, onto which a cannula may be connected. In an alternate embodiment, the supply vials are contained in base, coupling with the processing chamber in the handle once the hand-held is cradled in the base. Valves to control fluid path flow may be magnetically coupled and actuated in the base after aspiration is complete and the hand-held is inserted into the base.

The use of the device may in one embodiment involve three steps: aspiration, processing, and use of product. Aspiration may be minimally invasive due to the nature of small volume “micro-liposuction” and the use of a 16 or 14 gauge cannula. The hand-held design is intended to be small and easy to handle during the aspiration step. Upon completion of aspiration, the hand-held may be inserted into the base. The base may provide heat to the processor region of the hand-held to optimize the digestive process for a specified amount of time. An alternate embodiment includes an inductive charger to recharge the battery in the hand-held. In an alternate embodiment, the base contains the heater, supply vials, pressure and vacuum pumps, or any combination thereof, coupling with the hand-held when it is cradled in the base.

Cells may be concentrated and purified using a hollow-fiber bioreactor, in which cells are pushed through a dialysis membrane by positive or negative pressure. As the medium passes through the tubes, fluid and enzymes diffuse out of the fiber for disposal, while cells and cell debris pass through. This process may be repeated until the desired concentration of cells is achieved. Additional wash can be added to arrive at a preset dilution. An alternate embodiment separates and concentrates cells via centrifugation, in which the hand-held unit, or a portion thereof, is inserted into the base which in-turn doubles as a centrifuge. An alternate embodiment involves removing a portion of the hand-held after digestion is completed and inserting it into a separate centrifuge provided alongside of the base.

When processing is complete, product cells can be extracted via syringe, ready for use. In alternate embodiment, the cell product can be delivered by the device through a dedicated needle, at a specified delivery pressure and concentration. In another an alternate embodiment, the cell product can be extracted from the hand-held after processing via a syringe that contains an intermediate filter assembly. The negative pressure induced by withdrawing the syringe-plunger can pull the cells through the filter assembly before entering the syringe.

One embodiment is a single hand-held unit and a base. An alternate embodiment involves multiple hand-held units in a carousel style base, thereby giving an operator the option to harvest and process incremental volumes of fat, depending on their intended use. In another alternate embodiment, the carousel base doubles as a centrifuge.

### EXAMPLE

What follows is an illustrative example of a method of using one or more of the devices described herein.
Step 1: Doctor injects local anesthetic or tumescent fluid into patient.
Step 2: Micro-Lipo 10 cc's fat using 16 gauge cannula (1.3 mm) of device.
Step 3: Device pre-processes fat to remove connective tissue as suction aspiration continues.
Step 4: Lipo step completes, device is returned to base.
Step 5: Device runs through wash-rinse cycles.
Step 6: Device automatically initiates the digestion process.
Step 7: Temperature is raised to 37 C from heater block on base for digestion.
Step 8: Cells are automatically concentrated after digestion/rinse using vacuum.
Step 9: Final cell product is diluted according to set-point entered by doctor.
Step 10: In one embodiment, the doctor inserts syringe through product access point to retrieve final product. In an alternative embodiment, the doctor removes cannula and attaches needle to separate product port; detaches hand-held from suction tube; device uses onboard positive pressure source to expel product at optimal rate for surgery.

What is claimed is:

1. A device for harvesting, processing, and delivering a cell product, comprising a portable unit comprising:
a processing chamber;
a cannula with one end connected to the processing chamber in an airtight manner, and the other end open to the atmosphere;
a vacuum source connected to the processing chamber for creating a vacuum in the processing chamber when the vacuum source is activated, such that when the cannula is inserted into adipose tissue and the vacuum source is activated, the adipose tissue can be aspirated through the cannula into the processing chamber;
a connective tissue filter or screen within the processing chamber with a first side and a second side, suitable for separating connective tissue strands from adipose tissue which may arrive into the processing chamber through the cannula, wherein the connective tissue remains in the vicinity of the first side of the connective tissue filter or screen, and a composition comprising adipose cells is allowed to pass to the second side of the connective tissue filter or screen.

2. The device of claim 1, further comprising a rotor adjacent to the connective tissue filter or screen.

3. The device of claim 1, further comprising:
a digestion area within the processing chamber, in communication with the second side of the connective tissue filter or screen; and
a digestion filter or screen, one side of which is opening into the digestion area, configured to allow liquids to exit the digestion area, while retaining solid materials within the digestion area.

4. The device of claim 3, further comprising a product cell concentration chamber separated from the digestion area by the digestion filter or screen, wherein the digestion filter or screen is capable of permitting passage of free adipose cells, but retaining tissue structures within a cellular matrix.

5. The device of claim 3, further comprising:
a heater for heating a portion of the portable unit that includes the digestion area; and
circuitry for controlling the temperature of the heater.

6. The device of claim 3, further comprising a base adapted to hold the portable unit in a position where the digestion screen is horizontal

7. The device of claim 1, wherein the vacuum source is an evacuated vessel, further comprising a valve, wherein activating the vacuum source comprises opening the processing chamber to the evacuated vessel to create a vacuum within the processing chamber.

8. The device of claim 1, further comprising:
a liquid container suitable for holding liquids, comprising an inlet and an outlet such that when the container is full of liquid, a pressurized gas stream from the inlet will cause liquid to exit the outlet, wherein the outlet is in communication with the processing chamber;
a cylinder of pressurized gas comprising an outlet; and
a valve in communication with the inlet to the liquid container, and in communication with the outlet to the cylinder of pressurized gas.

9. A method for aspirating adipose tissue, comprising:
providing a portable device according to claim 1;
inserting a tip of the cannula into adipose tissue within a patient;
activating the vacuum source to induce a vacuum within the processing chamber, thereby causing a portion of the adipose tissue to move through the cannula into the processing chamber; and
in the processing chamber, separating connective tissue strands from said portion of the adipose tissue, such that the remaining adipose tissue passes to the second side of the connective tissue filter or screen.

10. The method of claim 9, wherein the portable device further comprises a digestion area within the processing chamber, in communication with the second side of the connective tissue filter or screen; further comprising the step of digesting said remaining adipose tissue in the digestion area.

11. The method of claim 10, wherein the portable device further comprises a product cell concentration chamber separated from the digestion area by a digestion filter or screen, one side of which is opening into the digestion area, configured to retain solid materials within the digestion area, further comprising the step of permitting free adipose cells to pass through the digestion filter or screen, after digestion occurs.

12. A method for aspirating adipose tissue, comprising:
providing a portable device comprising a cannula connected to a chamber, wherein the chamber comprises a pressure outlet in fluid communication with a valve, an wherein the valve is in fluid communication with an evacuated vessel;
inserting a tip of the cannula into adipose tissue within a patient; and
opening the valve to induce a vacuum within the chamber, thereby causing a portion of the adipose tissue to move through the cannula into the chamber.
